(19) United States
(12) Patent Application Publication
(10) Pub. No.: US 2026/0320256 A1
(43) Pub. Date: Oct. 15, 2026

(54) Digital Twin Based Tumor Growth Prediction Method And System

(71) Applicant: The First Affiliated Hospital of Jinan University (Guangzhou Overseas Chinese Hospital), Guangzhou (CN)

(72) Inventors: Shuixing Zhang, Guangzhou (CN); Bin Zhang, Guangzhou (CN); Lu Zhang, Guangzhou (CN); Jin Fang, Guangzhou (CN)

(21) Appl. No.: 19/419,619

(22) Filed: Dec. 15, 2025

(30) Foreign Application Priority Data
Apr. 14, 2025 (CN) 202510459093.2

(51) Int. Cl. G06F 30/27; G06T 7/00

(52) U.S. Cl. CPC G06F 30/27; G06T 7/0012; G06T 2207/30016; G06T 2207/30096

(57) ABSTRACT

A digital twin-based method and system for predicting tumor progression. The methodology involves: constructing a primary digital twin model integrating the patient's brain imaging data, clinical records, and physiological parameters; establishing a secondary digital twin model incorporating blood-brain barrier data, drug interaction characteristics, and dynamic microenvironment metrics. Through the primary model, initial treatment predictions are generated based on the treatment plan. The secondary model then produces updated treatment forecasts corresponding to the same protocol. By refining the model through iterative adjustments, a customized treatment plan is formulated incorporating the original predictions and subsequent refinements. The system achieves enhanced accuracy in tumor growth prediction by leveraging pharmacokinetic modeling, mechanistic simulations of drug interactions, and real-time monitoring of microenvironmental parameters.

### TECHNICAL FIELD

The present application relates to the field of tumor growth prediction technology, and more specifically, to a digital twin based tumor growth prediction method and system.

### BACKGROUND ART

Tumor growth prediction remains a pivotal challenge in precision medicine. Conventional approaches primarily rely on two-dimensional medical imaging analysis and static mathematical models, which struggle to dynamically simulate the spatiotemporal evolution of tumor cells and their interactions with complex microenvironments. Current technologies typically employ simplified physical equations or statistical learning models that overlook critical biological mechanisms such as tumor heterogeneity, angiogenesis, and immune evasion, resulting in limited predictive accuracy. Furthermore, traditional methods lack real-time adaptability and personalized customization capabilities, failing to dynamically adjust predictive models according to patients' treatment responses, thereby falling short of meeting the demands of clinical personalized therapy.

In recent years, the emergence of digital twin technology has provided a new paradigm for tumor growth prediction. By constructing three-dimensional dynamic simulation models of tumors and their microenvironments, digital twins integrate multimodal medical imaging, genetic sequencing, pathological reports, and other data to simulate tumor evolution under different therapeutic interventions. However, how existing digital twin models can effectively couple prediction results with clinical decision-making systems to achieve intelligent optimization of treatment plans remains a key challenge in current research. For instance, Patent CN119092136B (Application No.: CN202411586012.7) proposes a modeling method based on multimodal digital twin technology. This approach utilizes multimodal data from imaging, genetics, and clinical observations to generate predictive results for treatment efficacy through tumor digital twin simulations. However, the method in Patent CN119092136B relies solely on simplified pharmacokinetic models for predicting treatment outcomes, resulting in suboptimal intelligent optimization effects of the multimodal digital twin technology for treatment plans.

### SUMMARY OF THE INVENTION

The purpose of this application is to provide a digital twin-based tumor growth prediction method and system, which addresses the technical issue of relying solely on simplified pharmacokinetic models for predicting treatment outcomes and achieves the technical effect of improving the accuracy of tumor growth prediction by incorporating pharmacokinetic models, simulation of drug interaction mechanisms, and dynamic microenvironment parameters.

The digital twin-based tumor growth prediction method provided in the embodiments of this application comprises the following steps: constructing a digital twin database of multimodal pathological data for patients, where the multimodal pathological data includes brain imaging data, clinical data, patient physiological data, blood-brain barrier data, drug interaction data, and dynamic microenvironment data of the patients; constructing a first digital twin model using the brain imaging data, clinical data, and patient physiological data of the patients, with the first digital twin model reflecting the tumor growth direction and evolution speed characteristics of patients at different stages; and constructing a second digital twin model using the blood-brain barrier data, drug interaction data, and dynamic microenvironment data of the patients, with the second digital twin model reflecting the tumor treatment microenvironment characteristics of patients at different stages; determining a first treatment prediction result based on the patient's treatment plan using the first digital twin model; and determining a second treatment prediction result based on the patient's treatment plan using the second digital twin model; determining a revised treatment plan for the patient using a treatment plan revision model based on the patient's treatment plan, the first treatment prediction result, and the second treatment prediction result.

In one possible implementation approach, the method further comprises: determining the blood-brain barrier characteristics of the patient using a blood-brain barrier detection model based on the patient's brain imaging data and blood-brain barrier data; and determining a revised treatment plan for the patient using the treatment plan revision model based on the patient's treatment plan, the first treatment prediction result, the second treatment prediction result, and the blood-brain barrier characteristics.

In another possible implementation approach, the method further comprises: constructing drug concentration gradient brain imaging data corresponding to the patient's brain imaging data based on the patient's brain imaging data, blood-brain barrier data, and dynamic microenvironment data; constructing the first digital twin model of the patient's brain imaging data, clinical data, and patient physiological data includes: constructing the first digital twin model using the patient's drug concentration gradient brain imaging data, clinical data, and patient physiological data.

In yet another possible implementation approach, the method further comprises: identifying normal tissue regions and tumor-infiltrated regions in the patient's brain imaging data, and obtaining normal tissue characteristics corresponding to the normal tissue regions and infiltrated region characteristics corresponding to the tumor-infiltrated regions; constructing normal tissue drug concentration gradient brain imaging data for the patient based on the patient's normal tissue regions, normal tissue characteristics, blood-brain barrier data, and dynamic microenvironment data; constructing infiltrated region drug concentration gradient brain imaging data for the patient based on the patient's tumor-infiltrated regions, infiltrated region characteristics, blood-brain barrier data, and dynamic microenvironment data; constructing the first digital twin model of the patient's brain imaging data, clinical data, and patient physiological data includes: constructing the first digital twin model using the patient's normal tissue drug concentration gradient brain imaging data, infiltrated region drug concentration gradient brain imaging data, clinical data, and patient physiological data.

In still another possible implementation approach, the method further comprises: determining the patient's blood-brain barrier correction data, which represents changes in the blood-brain barrier data, using the first digital twin model based on the patient's blood-brain barrier data; constructing the second digital twin model of the patient's blood-brain barrier data, drug interaction data, and dynamic microenvironment data includes: constructing the second digital twin model using the patient's blood-brain barrier correction data, drug interaction data, and dynamic microenvironment data.

In another possible implementation approach, the method further comprises: acquiring normal region blood-brain barrier characteristics corresponding to normal tissue regions and infiltrated region blood-brain barrier characteristics corresponding to tumor-infiltrated regions; determining normal region blood-brain barrier data corresponding to normal tissue regions based on the patient's blood-brain barrier data, normal tissue regions in brain imaging data, and normal region blood-brain barrier characteristics; determining infiltrated region blood-brain barrier data corresponding to tumor-infiltrated regions based on the patient's blood-brain barrier data, tumor-infiltrated regions in brain imaging data, and infiltrated region blood-brain barrier characteristics; determining normal region blood-brain barrier correction data for the patient using the first digital twin model based on the patient's normal region blood-brain barrier data; determining infiltrated region blood-brain barrier correction data for the patient using the first digital twin model based on the patient's infiltrated region blood-brain barrier data; constructing the second digital twin model of the patient's blood-brain barrier data, drug interaction data, and dynamic microenvironment data includes: constructing the second digital twin model using the patient's normal region blood-brain barrier correction data, infiltrated region blood-brain barrier correction data, drug interaction data, and dynamic microenvironment data.

In yet another possible implementation approach, the method further comprises: acquiring multiple similar first digital twin models of the first digital twin model and multiple similar second digital twin models of the second digital twin model, obtaining treatment decision directions and subsequent first digital twin models corresponding to each treatment decision direction for the multiple similar first digital twin models; and obtaining treatment decision directions and subsequent second digital twin models corresponding to each treatment decision direction for the multiple similar second digital twin models; where the treatment decision directions include aggressive treatment approaches and conservative treatment approaches; among the multiple similar second digital twin models, identifying target similar second digital twin models corresponding to each similar first digital twin model based on parameter similarity between brain imaging data of the similar first digital twin models and blood-brain barrier data of the similar second digital twin models; when the treatment decision directions of a similar first digital twin model and its target similar second digital twin model are the same, designating the similar first digital twin model and its target similar second digital twin model as a predictive treatment decision model group, and using the treatment decision direction of the predictive treatment decision model group as the recommended treatment decision direction.

In still another possible implementation approach, the method further comprises: acquiring first subsequent tumor growth magnitudes corresponding to similar first digital twin models and second subsequent tumor growth magnitudes corresponding to target similar second digital twin models in each predictive treatment decision model group; determining the absolute difference between the first and second subsequent tumor growth magnitudes and the ratio of their sum as the subsequent tumor growth magnitude discrepancy; recommending the recommended treatment decision directions in ascending order based on the subsequent tumor growth magnitude discrepancies of multiple predictive treatment decision model groups.

In a further possible implementation approach, the method further comprises: acquiring first complication assessment values corresponding to similar first digital twin models and second complication assessment values corresponding to target similar second digital twin models in each predictive treatment decision model group; where the complication assessment values represent the impact of complications on the patient's quality of life; determining the absolute difference between the first and second complication assessment values and the ratio of their sum as the complication assessment value discrepancy; determining the sum of the subsequent tumor growth magnitude discrepancy and the complication assessment value discrepancy for each predictive treatment decision model group as the treatment direction comprehensive assessment value; recommending the recommended treatment decision directions in ascending order based on the treatment direction comprehensive assessment values of multiple predictive treatment decision model groups.

The embodiments of this application also provide a digital twin-based tumor growth prediction system, which includes units for executing the method described in any of the above embodiments.

Compared with the existing technologies, the beneficial effects of the embodiments of this application are as follows:

The embodiments of this application provide a digital twin-based tumor growth prediction method, which comprises the following steps: constructing a digital twin database of multimodal pathological data for patients, where the multimodal pathological data includes brain imaging data, clinical data, patient physiological data, blood-brain barrier data, drug interaction data, and dynamic microenvironment data of the patients; constructing a first digital twin model using the brain imaging data, clinical data, and patient physiological data of the patients, with the first digital twin model reflecting the tumor growth direction and evolution speed characteristics of patients at different stages; and constructing a second digital twin model using the blood-brain barrier data, drug interaction data, and dynamic microenvironment data of the patients, with the second digital twin model reflecting the tumor treatment microenvironment characteristics of patients at different stages; determining a first treatment prediction result based on the patient's treatment plan using the first digital twin model; and determining a second treatment prediction result based on the patient's treatment plan using the second digital twin model; determining a revised treatment plan for the patient using a treatment plan revision model based on the patient's treatment plan, the first treatment prediction result, and the second treatment prediction result. In the embodiments of this application, the first digital twin model can reflect the tumor growth direction and evolution speed characteristics of patients at different stages, while the second digital twin model can reflect the tumor treatment microenvironment characteristics of patients at different stages. The treatment plan revision model can revise the first and second treatment prediction results, enabling the determination of a tumor treatment plan based on the tumor growth direction and evolution speed characteristics, as well as the treatment microenvironment characteristics. Furthermore, it allows for comparative verification of the treatment plan based on the treatment effects indicated by the first and second treatment prediction results, thereby improving the simulation effect of brain tumor treatment outcomes.

### BRIEF DESCRIPTION OF ACCOMPANY DRAWINGS

To provide a clearer illustration of the technical solutions in the embodiments of this application, a brief introduction to the accompanying drawings required for describing the embodiments or the existing technologies will be given below. It is evident that the drawings described below merely represent some embodiments of this application, and for those skilled in the art, other drawings can also be obtained based on these without the need for creative effort.

FIG. 1 is a schematic flowchart of the first digital twin-based tumor growth prediction method provided in an embodiment of this application;

FIG. 2 is a schematic workflow diagram of the first digital twin-based tumor growth prediction method provided in an embodiment of this application;

FIG. 3 is a schematic flowchart of the second digital twin-based tumor growth prediction method provided in an embodiment of this application;

FIG. 4 is a schematic flowchart of the third digital twin-based tumor growth prediction method provided in an embodiment of this application;

FIG. 5 is a tissue schematic diagram of normal tissue regions and tumor-infiltrated regions provided in an embodiment of this application;

FIG. 6 is a schematic flowchart of the fourth digital twin-based tumor growth prediction method provided in an embodiment of this application;

FIG. 7 is a schematic flowchart of the fifth digital twin-based tumor growth prediction method provided in an embodiment of this application;

FIG. 8 is a schematic flowchart of the sixth digital twin-based tumor growth prediction method provided in an embodiment of this application;

FIG. 9 is a schematic flowchart of the seventh digital twin-based tumor growth prediction method provided in an embodiment of this application;

FIG. 10 is a schematic logic structure diagram of a digital twin-based tumor growth prediction system provided in an embodiment of this application.

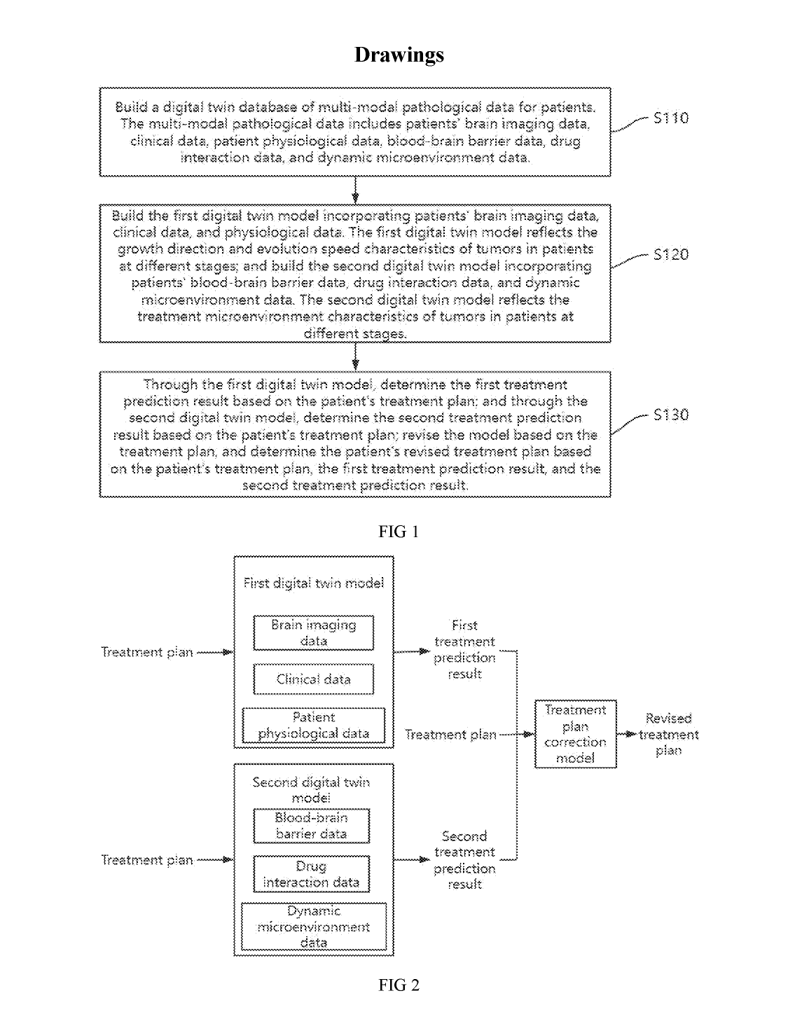

### SPECIFIC EMBODIMENT OF THE INVENTION

It should be understood that when used in the specification and the appended claims of this application, the term "comprise" indicates the presence of described characteristics, entities, steps, operations, elements, and/or components, but does not preclude the presence or addition of one or more other characteristics, entities, steps, operations, elements, components, and/or groups thereof.

It should also be understood that the term "and/or" used in the specification and the appended claims of this application refers to any combination and all possible combinations of the items listed in association, including these combinations. Similarly, the phrase "if" as used in the specification and the appended claims of this application can be interpreted, depending on the context, as "when...", "once", "in response to determining", or "in response to detecting". Likewise, the phrases "if determined" or "if [the described condition or event] is detected" can be interpreted, depending on the context, to mean "once determined", "in response to determining", "once [the described condition or event] is detected", or "in response to detecting [the described condition or event]".

Additionally, in the descriptions of the specification and the appended claims of this application, the terms "first," "second," "third," etc., are used solely for distinguishing between descriptions and should not be interpreted as indicating or implying relative importance.

References to "one embodiment" or "some embodiments" in the specification of this application indicate that the specific characteristics, structures, or characteristics described in connection with that embodiment are included in one or more embodiments of this application. Thus, statements such as "in one embodiment," "in some embodiments," "in other embodiments," and "in additional embodiments" appearing elsewhere in this specification do not necessarily refer to the same embodiment but rather mean "one or more but not all embodiments," unless otherwise specifically emphasized. The terms "comprise," "include," "have," and their variations all mean "include but are not limited to," unless otherwise specifically emphasized.

Existing modeling methods based on multimodal digital twin technology can perform multimodal digital twin simulations of tumors using multimodal data from imaging, genetics, and clinical sources to generate predictive results for treatment plan effectiveness. However, current modeling methods based on multimodal digital twin technology rely solely on simplified pharmacokinetic models to predict treatment outcomes, resulting in suboptimal intelligent optimization of treatment plans by the multimodal digital twin technology.

For the above reasons, the embodiments of this application provide a digital twin-based tumor growth prediction method, which comprises the following steps: constructing a digital twin database of multimodal pathological data for patients, where the multimodal pathological data includes brain imaging data, clinical data, patient physiological data, blood-brain barrier data, drug interaction data, and dynamic microenvironment data of the patients; constructing a first digital twin model using the brain imaging data, clinical data, and patient physiological data of the patients, where the first digital twin model reflects the tumor growth direction and evolution speed characteristics of patients at different stages; constructing a second digital twin model using the blood-brain barrier data, drug interaction data, and dynamic microenvironment data of the patients, where the second digital twin model reflects the tumor treatment microenvironment characteristics of patients at different stages; determining a first treatment prediction result based on the patient's treatment plan using the first digital twin model; determining a second treatment prediction result based on the patient's treatment plan using the second digital twin model; and determining a revised treatment plan for the patient using a treatment plan revision model based on the patient's treatment plan, the first treatment prediction result, and the second treatment prediction result. In the embodiments of this application, the first digital twin model can reflect the tumor growth direction and evolution speed characteristics of patients at different stages, while the second digital twin model can reflect the tumor treatment microenvironment characteristics of patients at different stages. The treatment plan revision model can revise the first and second treatment prediction results, enabling the determination of a tumor treatment plan based on the tumor growth direction and evolution speed characteristics, as well as the treatment microenvironment characteristics. Furthermore, it allows for comparative verification of the treatment plan based on the treatment effects indicated by the first and second treatment prediction results, thereby improving the simulation effect of brain tumor treatment outcomes.

In some scenarios, the digital twin-based tumor growth prediction method provided in the embodiments of this application can be applied to the treatment of brain tumors, enhancing the digital twin simulation effect in brain tumor treatment and improving the predictive effect of brain tumor treatment.

The following provides a detailed explanation of a digital twin-based tumor growth prediction method provided in the embodiments of this application through specific examples.

FIG. 1 is a schematic flowchart of the first digital twin-based tumor growth prediction method provided in the embodiments of this application. As shown in FIG. 1, this method comprises steps S110 to S130, which will be elaborated upon below.

S110: Construct a digital twin database of multimodal pathological data for patients, where the multimodal pathological data includes brain imaging data, clinical data, patient physiological data, blood-brain barrier data, drug interaction data, and dynamic microenvironment data of the patients.

Given that current modeling methods based on multimodal digital twin technology rely solely on simplified pharmacokinetic models to predict treatment outcomes, resulting in suboptimal intelligent optimization of treatment plans, in the embodiments of this application, to avoid predicting treatment outcomes solely through simplified pharmacokinetic models, a digital twin database of multimodal pathological data for patients is first constructed. This multimodal pathological data includes brain imaging data, clinical data, patient physiological data, blood-brain barrier data, drug interaction data, and dynamic microenvironment data of the patients, thereby enhancing the accuracy of predicting treatment outcomes through the digital twin database, which includes blood-brain barrier data, drug interaction data, and dynamic microenvironment data, and avoiding the issue of poor tumor treatment simulation effects through simplified pharmacokinetic models.

For example, brain imaging data can be acquired through magnetic resonance imaging (MRI), which generates high-resolution brain structure images using magnetic fields and radio waves.Brain imaging data can also be rapidly obtained through computed tomography (CT) for quick cross-sectional brain imaging, suitable for preliminary diagnosis in emergency situations. Additionally, positron emission tomography (PET) can be used in conjunction with radioactive tracers to detect tumor metabolic activity (e.g., FDG-PET) for brain imaging data collection.

For example, clinical data can be acquired through electronic health records (EHR), specifically by extracting patient medical history, diagnostic results, and treatment records from hospital HIS systems for clinical data collection. Structured forms such as standardized tables (e.g., CRFs, case report forms) can also be used to record patient symptoms and medication history for clinical data acquisition.

For example, patient physiological data can be obtained by measuring vital signs using conventional detection equipment such as sphygmomanometers, electrocardiographs, and pulse oximeters. Patient physiological data can also be derived from laboratory tests such as blood tests (complete blood count, liver and kidney function) and urinalysis.

For example, blood-brain barrier data can be acquired through MRI enhanced scanning by assessing blood-brain barrier permeability (e.g., peritumoral edema) after injecting gadolinium contrast agents. Blood-brain barrier data can also be obtained through lumbar puncture (LP) by detecting proteins and glucose in cerebrospinal fluid (CSF) to indirectly reflect blood-brain barrier function. Furthermore, blood-brain barrier data can be acquired through biomarker detection of S100B protein and vascular endothelial growth factor (VEGF) in blood or cerebrospinal fluid. Molecular imaging techniques such as ultrasmall superparamagnetic iron oxide (USPIO) nanoparticle MRI can also be used to assess blood-brain barrier integrity.

For example, drug interaction data can be acquired from drug databases such as Micromedex, Lexicomp, and UpToDate, which provide information on interactions between drugs. Drug interaction data can also be supplemented by literature searches to track the latest research and cover rare interactions not included in the databases.

For example, dynamic microenvironment data can be obtained through tumor biopsy by analyzing tumor cells, immune cells, and angiogenesis (e.g., immunohistochemistry, gene sequencing) in tissue samples. Dynamic microenvironment data can also be detected through liquid biopsy by examining circulating tumor DNA (ctDNA) and exosomes to monitor dynamic changes in the microenvironment.

S120: Construct a first digital twin model using the brain imaging data, clinical data, and patient physiological data of the patients, where the first digital twin model reflects the tumor growth direction and evolution speed characteristics of patients at different stages. Additionally, construct a second digital twin model using the blood-brain barrier data, drug interaction data, and dynamic microenvironment data of the patients, where the second digital twin model reflects the tumor treatment microenvironment characteristics of patients at different stages.

After obtaining the digital twin database, a first digital twin model can be constructed using the brain imaging data, clinical data, and patient physiological data of the patients. Since brain imaging data, clinical data, and patient physiological data can predict tumor growth processes at a macroscopic level, the first digital twin model can reflect the tumor growth direction and evolution speed characteristics of patients at different stages by extracting tumor volume changes through image segmentation techniques and predicting growth speed in conjunction with clinical chemotherapy regimens.

After obtaining the digital twin database, a second digital twin model can also be constructed using the blood-brain barrier data, drug interaction data, and dynamic microenvironment data of the patients, enabling the second digital twin model to reflect the tumor treatment microenvironment characteristics of patients at different stages. The blood-brain barrier data, drug interaction data, and dynamic microenvironment data in the second digital twin model are more suitable for microscopic mechanism modeling and can achieve real-time dynamic updates, enabling multiscale correlation analysis from molecules (e.g.,drug targets) to tissues (e.g., tumor vascular density). They can also optimize treatment for patient-specific microenvironments (e.g., immunosuppressive tumor microenvironments) and facilitate the design of combined treatment plans, such as the combination of immune checkpoint inhibitors and anti-angiogenic drugs.

S130: Determine a first treatment prediction result based on the patient's treatment plan using the first digital twin model. Determine a second treatment prediction result based on the patient's treatment plan using the second digital twin model. Determine a revised treatment plan for the patient using a treatment plan revision model based on the patient's treatment plan, the first treatment prediction result, and the second treatment prediction result.

After obtaining the first and second digital twin models, a first treatment prediction result can be determined based on the patient's treatment plan using the first digital twin model. The first treatment prediction result can predict treatment outcomes based on the tumor growth direction and evolution speed characteristics at different stages.

After obtaining the first and second digital twin models, a second treatment prediction result can also be determined based on the patient's treatment plan using the second digital twin model. The second treatment prediction result can predict treatment outcomes based on the tumor treatment microenvironment characteristics at different stages.

Since the first treatment prediction result represents the treatment outcomes derived from the tumor growth direction and evolution speed characteristics at different stages, and the second treatment prediction result represents the treatment outcomes derived from the tumor treatment microenvironment characteristics at different stages, after obtaining the first and second treatment prediction results, a revised treatment plan for the patient can be determined using a treatment plan revision model based on the patient's treatment plan, the first treatment prediction result, and the second treatment prediction result. This achieves the purpose of revising the treatment measures in the patient's treatment plan based on the patient's treatment plan, the first treatment prediction result, and the second treatment prediction result.

For example, the treatment plan revision model can be a neural network model trained using labeled patient treatment plans, first treatment prediction results, second treatment prediction results, and labeled revised treatment plans for patients. The labeled revised treatment plans for patients are used to correct conflicting content between the labeled patient treatment plans and the first and second treatment prediction results, achieving the purpose of revising the patient's treatment plan based on the first and second treatment prediction results and improving the credibility and treatment effectiveness of the patient's treatment plan.

The beneficial effects of the above implementation approach are as follows: Firstly, the first digital twin model can reflect the tumor growth direction and evolution speed characteristics of patients at different stages. Secondly, the blood-brain barrier data, drug interaction data, and dynamic microenvironment data in the second digital twin model are more suitable for microscopic mechanism modeling. By simulating the tumor development process through characteristics in different dimensions, the digital simulation effect of the tumor treatment process is improved.

The beneficial effects of the above implementation approach also lie in the fact that after obtaining the first treatment prediction result and the second treatment prediction result, a treatment plan correction model can be used to determine the patient's corrected treatment plan based on the patient's treatment plan, the first treatment prediction result, and the second treatment prediction result. This enables a comprehensive correction of the treatment plan according to the tumor growth direction and evolution speed at different stages as well as the treatment microenvironment characteristics, further enhancing the therapeutic effect of the patient's treatment plan on the tumor.

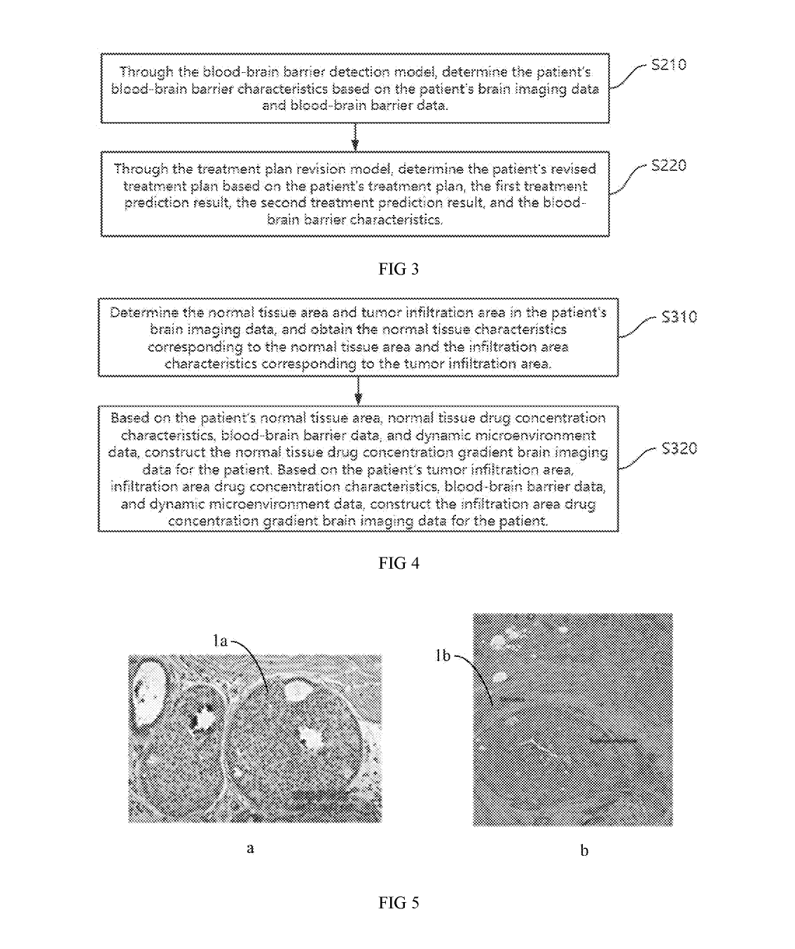

FIG. 3 is a flowchart diagram of a second tumor growth prediction method based on digital twins provided in an embodiment of this application. As shown in FIG. 3, the above method also includes S210 to S220, which are described in detail below.

S210: Determine the patient's blood-brain barrier characteristics through a blood-brain barrier detection model based on the patient's brain imaging data and blood-brain barrier data.

Both the patient's brain imaging data in the first digital twin model and the blood-brain barrier data in the second digital twin model can represent the patient's blood-brain barrier characteristics at the imaging level and the microenvironment level. In this embodiment of the application, the patient's blood-brain barrier characteristics can be determined through a blood-brain barrier detection model based on the patient's brain imaging data and blood-brain barrier data. Subsequently, the patient's treatment plan can be further comprehensively adjusted according to the patient's blood-brain barrier characteristics.

For example, brain imaging data (e.g., MRI, CT) provides spatial localization and anatomical structure information (e.g., tumor location, edema range), while blood-brain barrier data (e.g., dynamic contrast enhancement parameters, S100B protein levels) reflects functional status (e.g., permeability, integrity).

S220: Determine the patient's corrected treatment plan through a treatment plan correction model based on the patient's treatment plan, the first treatment prediction result, the second treatment prediction result, and the blood-brain barrier characteristics.

In tumor treatment, blood-brain barrier characteristics have a significant impact on the tumor treatment process. Therefore, in this embodiment of the application, the patient's corrected treatment plan can be determined through a treatment plan correction model based on the patient's treatment plan, the first treatment prediction result, the second treatment prediction result, and the blood-brain barrier characteristics. This achieves a further correction of the patient's treatment plan in combination with the blood-brain barrier characteristics and is suitable for individualized treatment of complex central nervous system diseases (e.g., malignant brain tumors, autoimmune encephalitis).

The beneficial effects of the above implementation approach are that the blood-brain barrier characteristics represent the patient's blood-brain barrier characteristics at the imaging level and the microenvironment level. This enables a further correction of the patient's treatment plan in combination with the blood-brain barrier characteristics, which is suitable for individualized treatment of complex central nervous system diseases and improves the digital simulation effect of the treatment of brain tumors.

In some implementation approaches, the above method also includes: constructing drug concentration gradient brain imaging data corresponding to the patient's brain imaging data based on the patient's brain imaging data, blood-brain barrier data, and dynamic microenvironment data.

During brain tumor treatment, brain imaging data, blood-brain barrier data, and dynamic microenvironment data can affect the drug concentration in different tissue regions. In this embodiment of the application, drug concentration gradient brain imaging data corresponding to the patient's brain imaging data can be constructed based on the patient's brain imaging data, blood-brain barrier data, and dynamic microenvironment data. The drug concentration gradient brain imaging data represents the drug concentration information in different tissue regions, thereby further improving the simulation effect of the patient's tumor treatment process according to the patient's drug concentration gradient brain imaging data.

For example, when constructing drug concentration gradient brain imaging data corresponding to the patient's brain imaging data based on the patient's brain imaging data, blood-brain barrier data, and dynamic microenvironment data, first, imaging data such as MRI/CT (structure), PET (metabolism), and DCE-MRI (blood flow/permeability) can be spatially aligned to a unified coordinate system (e.g., the MNI standard space). Blood-brain barrier-related molecular markers (e.g.,VEGF, MMP-9) and gene expression characteristics (e.g. ,ABCB1 transporter) can be superimposed onto the imaging space through immunohistochemistry or in situ hybridization results. Parameters such as blood flow velocity (PWI-MRI), interstitial pressure (TIP), and pH value (1H-MRS) can be extracted to construct voxel-level microenvironment feature vectors, thereby obtaining drug concentration gradient brain imaging data.

In some implementation approaches, constructing the first digital twin model of the patient's brain imaging data, clinical data, and patient physiological data in the above S120 includes: constructing the first digital twin model of the patient's drug concentration gradient brain imaging data, clinical data, and patient physiological data.

After obtaining the drug concentration gradient brain imaging data, the first digital twin model of the patient's drug concentration gradient brain imaging data, clinical data, and patient physiological data can be constructed, enabling the first digital twin model to make accurate predictions about the tumor treatment effect in combination with the drug concentration gradient brain imaging data and improving the digital simulation effect of the first digital twin model for tumor treatment.

The beneficial effects of the above implementation approach are that, based on the first digital twin model that can represent the tumor growth direction and evolution speed characteristics at different stages, accurate predictions about the tumor treatment effect can be made in combination with the drug concentration gradient brain imaging data, improving the digital simulation effect of the first digital twin model for tumor treatment.

FIG. 4 is a flowchart diagram of a third tumor growth prediction method based on digital twins provided in an embodiment of this application. As shown in FIG. 4, the above method also includes S310 to S320, which are described in detail below.

S310: Determine the normal tissue regions and tumor-infiltrated regions in the patient's brain imaging data, and obtain the normal tissue characteristics corresponding to the normal tissue regions and the infiltrated region characteristics corresponding to the tumor-infiltrated regions.

In the first digital twin model, due to differences in the integrity of the blood-brain barrier, tumor microenvironment characteristics, and drug delivery mechanisms between normal tissue regions and tumor-infiltrated regions, to further improve the prediction effect of the first digital twin model on the tumor growth state, digital simulations can be performed separately for the normal tissue regions and tumor-infiltrated regions in this embodiment of the application. First, the normal tissue regions and tumor-infiltrated regions in the patient's brain imaging data can be determined, and the normal tissue drug concentration characteristics corresponding to the normal tissue regions and the infiltrated region drug concentration characteristics corresponding to the tumor-infiltrated regions can be obtained. The normal tissue drug concentration characteristics represent the drug diffusion characteristics in the normal tissue regions, while the infiltrated region drug concentration characteristics represent the drug diffusion characteristics in the infiltrated regions.

FIG. 5 is a tissue diagram of normal tissue regions and tumor-infiltrated regions provided in an embodiment of this application. As shown in FIG. 5a, tumor cells 1a in the normal tissue region are located within the tissue epithelium and have not penetrated the tissue basement membrane, while tumor cells 1b in the tumor-infiltrated region have infiltrated outward into the surrounding tissue, forming an infiltrative tumor.

For example, the normal tissue drug concentration characteristics corresponding to the normal tissue regions and the infiltrated region drug concentration characteristics corresponding to the tumor-infiltrated regions can be obtained by testing tissue samples or from a tissue sample database.

S320: Construct the normal tissue drug concentration gradient brain imaging data of the patient based on the patient's normal tissue regions, normal tissue drug concentration characteristics, blood-brain barrier data, and dynamic microenvironment data. Construct the infiltrated region drug concentration gradient brain imaging data of the patient based on the patient's tumor-infiltrated regions, infiltrated region drug concentration characteristics, blood-brain barrier data, and dynamic microenvironment data.

After obtaining the normal tissue drug concentration characteristics, the normal tissue drug concentration gradient brain imaging data of the patient can be constructed based on the patient's normal tissue regions, normal tissue drug concentration characteristics, blood-brain barrier data, and dynamic microenvironment data. The normal tissue drug concentration gradient brain imaging data represents the drug concentration data at each position in the normal tissue regions.

After obtaining the infiltrated region drug concentration characteristics, the infiltrated region drug concentration gradient brain imaging data of the patient can be constructed based on the patient's tumor-infiltrated regions, infiltrated region drug concentration characteristics, blood-brain barrier data, and dynamic microenvironment data. The infiltrated region drug concentration gradient brain imaging data represents the drug concentration data at each position in the infiltrated regions.

In some implementation approaches, constructing the first digital twin model of the patient's brain imaging data, clinical data, and patient physiological data in the above S120 includes: constructing the first digital twin model of the patient's normal tissue drug concentration gradient brain imaging data, infiltrated region drug concentration gradient brain imaging data, clinical data, and patient physiological data.

After obtaining the normal tissue drug concentration gradient brain imaging data and the infiltrated region drug concentration gradient brain imaging data, the first digital twin model of the patient's normal tissue drug concentration gradient brain imaging data, infiltrated region drug concentration gradient brain imaging data, clinical data, and patient physiological data can be constructed. Subsequently, tumor treatment effects can be simulated according to the drug concentrations in the normal tissue regions and infiltrated regions based on the normal tissue drug concentration gradient brain imaging data and the infiltrated region drug concentration gradient brain imaging data, improving the digital simulation effect of the first digital twin model for tumor treatment.

The beneficial effects of the above implementation approach are that tumor treatment effects can be simulated according to the drug concentrations in the normal tissue regions and infiltrated regions based on the normal tissue drug concentration gradient brain imaging data and the infiltrated region drug concentration gradient brain imaging data. This improves the digital simulation effect of the first digital twin model for tumor treatment based on the first digital twin model that can represent the tumor growth direction and evolution speed characteristics at different stages.

In some implementation approaches, the aforementioned method also includes the following step: determining a patient's blood-brain barrier correction data based on the patient's blood-brain barrier data through the first digital twin model, where the blood-brain barrier correction data represents the change information of the blood-brain barrier data.

To enhance the accuracy of the blood-brain barrier data, in the embodiments of this application, the first digital twin model can also be utilized. Since the first digital twin model incorporates the growth direction and evolution speed characteristics of tumors at different stages, and these characteristics can, to a certain extent, represent the changes in blood-brain barrier data, it is possible to determine a patient's blood-brain barrier correction data based on the patient's blood-brain barrier data. The blood-brain barrier correction data represents the change information of the blood-brain barrier data, thereby enabling the improvement of the simulation effect of the second digital twin model through the blood-brain barrier correction data.

In some implementation approaches, in the aforementioned S120, constructing the second digital twin model of a patient's blood-brain barrier data, drug interaction data, and dynamic microenvironment data includes constructing the second digital twin model of a patient's blood-brain barrier correction data, drug interaction data, and dynamic microenvironment data.

After obtaining the blood-brain barrier correction data, the second digital twin model can be constructed using a patient's blood-brain barrier correction data, drug interaction data, and dynamic microenvironment data. Subsequently, the inclusion of the blood-brain barrier correction data in the second digital twin model can further enhance the effectiveness of microscopic mechanism modeling and improve the digital simulation of the tumor treatment process.

The beneficial effects of the aforementioned implementation approaches are that the inclusion of the blood-brain barrier correction data in the second digital twin model further enhances the effectiveness of microscopic mechanism modeling and improves the digital simulation of the tumor treatment process.

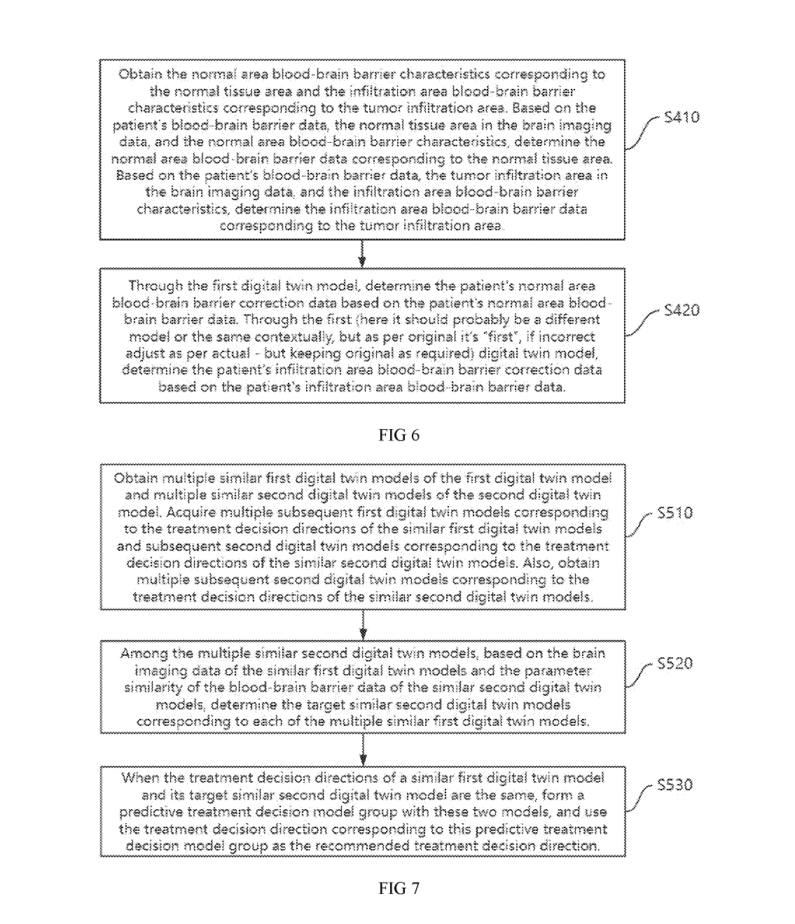

FIG. 6 is a flowchart of the fourth tumor growth prediction method based on digital twins provided in an embodiment of this application. As shown in FIG. 6, the aforementioned method further includes steps S410 to S420, which will be specifically described below.

S410: Obtain the normal-region blood-brain barrier characteristics corresponding to the normal tissue region and the infiltrated-region blood-brain barrier characteristics corresponding to the tumor-infiltrated region. Based on the patient's blood-brain barrier data, the normal tissue region in brain imaging data, and the normal-region blood-brain barrier characteristics, determine the normal-region blood-brain barrier data corresponding to the normal tissue region. Based on the patient's blood-brain barrier data, the tumor-infiltrated region in brain imaging data, and the infiltrated-region blood-brain barrier characteristics, determine the infiltrated-region blood-brain barrier data corresponding to the tumor-infiltrated region.

After identifying the normal tissue region and the tumor-infiltrated region, since these regions have distinct blood-brain barrier characteristics, the simulation effect of the second digital twin model on the blood-brain barrier in the normal tissue region and the tumor-infiltrated region can be further improved.

To further enhance the blood-brain barrier simulation effect of the first digital twin model for the normal tissue region and the tumor-infiltrated region, the normal-region blood-brain barrier characteristics corresponding to the normal tissue region and the infiltrated-region blood-brain barrier characteristics corresponding to the tumor-infiltrated region can be obtained. Subsequently, the blood-brain barrier simulation effect of the second digital twin model can be refined based on these characteristics.

For example, the normal-region blood-brain barrier characteristics corresponding to the normal tissue region and the infiltrated-region blood-brain barrier characteristics corresponding to the tumor-infiltrated region can be determined through dynamic contrast-enhanced MRI measurements. For instance, the normal-region blood-brain barrier characteristics can reflect the blood-brain barrier permeability by measuring the transfer coefficient (Ktrans) of the contrast agent leaking from the vascular lumen into the tissue interstitial space in the normal tissue region.

After obtaining the normal-region blood-brain barrier characteristics and the infiltrated-region blood-brain barrier characteristics, the normal-region blood-brain barrier data corresponding to the normal tissue region can be determined based on the patient's blood-brain barrier data, the normal tissue region in brain imaging data, and the normal-region blood-brain barrier characteristics, thereby achieving digital simulation of the normal-region blood-brain barrier data.

Similarly, after obtaining the normal-region blood-brain barrier characteristics and the infiltrated-region blood-brain barrier characteristics, the infiltrated-region blood-brain barrier data corresponding to the tumor-infiltrated region can be determined based on the patient's blood-brain barrier data, the tumor-infiltrated region in brain imaging data, and the infiltrated-region blood-brain barrier characteristics, thereby achieving digital simulation of the infiltrated-region blood-brain barrier data.

S420: Utilize the first digital twin model to determine the patient's normal-region blood-brain barrier correction data based on the patient's normal-region blood-brain barrier data. Similarly, use the first digital twin model to determine the patient's infiltrated-region blood-brain barrier correction data based on the patient's infiltrated-region blood-brain barrier data.

After obtaining the normal-region blood-brain barrier data, the first digital twin model can be employed to determine the patient's normal-region blood-brain barrier correction data based on this data, thereby achieving correction of the normal-region blood-brain barrier data.

Likewise, after acquiring the infiltrated-region blood-brain barrier data, the first digital twin model can be used to determine the patient's infiltrated-region blood-brain barrier correction data based on this data, enabling correction of the infiltrated-region blood-brain barrier data.

In some implementation approaches, constructing the second digital twin model of the patient's blood-brain barrier data, drug interaction data, and dynamic microenvironment data in the aforementioned S120 includes constructing the second digital twin model of the patient's normal-region blood-brain barrier correction data, infiltrated-region blood-brain barrier correction data, drug interaction data, and dynamic microenvironment data.

After obtaining the normal-region blood-brain barrier correction data and the infiltrated-region blood-brain barrier correction data, the second digital twin model can be constructed using the patient's normal-region blood-brain barrier correction data, infiltrated-region blood-brain barrier correction data, drug interaction data, and dynamic microenvironment data. This enables the second digital twin model to enhance the simulation effect of the blood-brain barrier in both the normal tissue region and the tumor-infiltrated region.

The beneficial effects of the aforementioned implementation approaches are as follows: Since the normal tissue region and the tumor-infiltrated region have distinct blood-brain barrier characteristics, by constructing normal-region blood-brain barrier data and infiltrated-region blood-brain barrier data for these regions and then correcting the blood-brain barrier data, and further constructing the second digital twin model using the patient's normal-region blood-brain barrier correction data, infiltrated-region blood-brain barrier correction data, drug interaction data, and dynamic microenvironment data, the second digital twin model can improve the simulation effect of the blood-brain barrier in both the normal tissue region and the tumor-infiltrated region, thereby enhancing the digital simulation effect of tumor treatment.

FIG. 7 is a flowchart of the fifth tumor growth prediction method based on digital twins provided in an embodiment of this application. As shown in FIG. 7, the aforementioned method further includes steps S510 to S530, which will be specifically described below.

S510: Obtain multiple similar first digital twin models of the first digital twin model and multiple similar second digital twin models of the second digital twin model. Acquire the treatment decision directions corresponding to each of the multiple similar first digital twin models and the subsequent first digital twin models associated with these treatment decision directions. Similarly, obtain the treatment decision directions corresponding to each of the multiple similar second digital twin models and the subsequent second digital twin models associated with these treatment decision directions. Here, the treatment decision directions include aggressive treatment approaches and conservative treatment approaches.

When conducting digital twin simulations for tumors, multiple similar first digital twin models of the first digital twin model and multiple similar second digital twin models of the second digital twin model can be obtained. This enables further enhancement of the simulation effect on the tumor treatment process based on these similar models.

During digital twin simulations, it is also possible to acquire the treatment decision directions corresponding to each of the multiple similar first digital twin models and the subsequent first digital twin models associated with these directions. The subsequent first digital twin models represent the digital twin models of the similar first digital twin models after tumor development during treatment, thereby facilitating the simulation of the subsequent development of the first digital twin model based on these subsequent models.

Similarly, the treatment decision directions corresponding to each of the multiple similar second digital twin models and the subsequent second digital twin models associated with these directions can be obtained. The subsequent second digital twin models can then be used to simulate the subsequent tumor development of the second digital twin model.

It should be noted that the treatment decision directions include aggressive treatment approaches and conservative treatment approaches, enabling auxiliary judgment of the treatment decision directions based on either aggressive or conservative treatment methods to achieve more accurate determination of the treatment direction.

S520: Among the multiple similar second digital twin models, determine the target similar second digital twin models corresponding to each of the multiple similar first digital twin models based on the parameter similarity between the brain imaging data of the similar first digital twin models and the blood-brain barrier data of the similar second digital twin models.

After obtaining multiple similar first digital twin models of the first digital twin model and multiple similar second digital twin models of the second digital twin model, to determine the optimal set of similar models for simulating the first and second digital twin models, the target similar second digital twin models corresponding to each of the multiple similar first digital twin models can be identified. This identification is based on the parameter similarity between the brain imaging data of the similar first digital twin models and the blood-brain barrier data of the similar second digital twin models, ensuring that the blood-brain barrier data of each target similar second digital twin model is similar to the brain imaging data of its corresponding similar first digital twin model.

For example, when determining the target similar second digital twin models corresponding to each of the multiple similar first digital twin models based on the parameter similarity between the brain imaging data of the similar first digital twin models and the blood-brain barrier data of the similar second digital twin models, a parameter similarity comparison model can be employed. This model extracts the parameter similarity between the brain imaging data of the similar first digital twin models and the blood-brain barrier data of the similar second digital twin models, and then identifies the target similar second digital twin models corresponding to each of the multiple similar first digital twin models in descending order of parameter similarity.

For instance, the parameter similarity comparison model can be trained based on labeled brain imaging data of similar first digital twin models, blood-brain barrier data of similar second digital twin models, and their corresponding parameter similarities.

S530: When the treatment decision directions of the similar first digital twin model and the target similar second digital twin model are the same, the similar first digital twin model and the target similar second digital twin model are grouped as a predictive treatment decision model set, and the treatment decision direction corresponding to this predictive treatment decision model set is designated as the recommended treatment decision direction.

After obtaining the similar first digital twin model and the target similar second digital twin model, if their treatment decision directions are identical, it indicates that both models represent subsequent digital twin models derived through the same treatment approach. In this case, the similar first digital twin model and the target similar second digital twin model can be grouped as a predictive treatment decision model set, and the treatment decision direction corresponding to this set can be recommended as the treatment approach, thereby providing a recommendation for the treatment decision direction.

The beneficial effects of the aforementioned implementation methods are as follows: Firstly, by separately identifying similar first digital twin models and similar second digital twin models and simulating the tumor treatment process based on these models, the simulation effect of the tumor treatment process is enhanced. Secondly, by determining similar first digital twin models and target similar second digital twin models with identical treatment decision directions and recommending these directions as treatment approaches, the accuracy of treatment decision recommendations is improved.

FIG. 8 is a flowchart of the sixth tumor growth prediction method based on digital twins provided in an embodiment of this application. As shown in FIG. 8, the aforementioned method further includes steps S610 to S620, which will be specifically described below.

S610: Obtain the first subsequent tumor growth magnitude corresponding to the similar first digital twin model and the second subsequent tumor growth magnitude corresponding to the target similar second digital twin model within each predictive treatment decision model set.

When simulating the tumor treatment process, it is further possible to obtain the first subsequent tumor growth magnitude corresponding to the similar first digital twin model and the second subsequent tumor growth magnitude corresponding to the target similar second digital twin model within each predictive treatment decision model set. Based on these magnitudes, the effectiveness of tumor treatment can be evaluated.

For example, the first subsequent tumor growth magnitude and the second subsequent tumor growth magnitude can represent the respective increases in area within the tumor region images of the similar first digital twin model and the target similar second digital twin model.

S620: Determine the absolute value of the difference between the first subsequent tumor growth magnitude and the second subsequent tumor growth magnitude, as well as the ratio of this difference to the sum of the first and second subsequent tumor growth magnitudes, and designate this ratio as the subsequent tumor growth magnitude discrepancy. Recommend the recommended treatment decision directions in ascending order based on the subsequent tumor growth magnitude discrepancies of multiple predictive treatment decision model sets.

After obtaining the first subsequent tumor growth magnitude and the second subsequent tumor growth magnitude, the absolute value of the difference between them, along with the ratio of this difference to their sum, can be calculated and designated as the subsequent tumor growth magnitude discrepancy. This discrepancy represents the ratio of the difference in the increase magnitudes corresponding to the first and second subsequent tumor growth magnitudes to the sum of these increase magnitudes, effectively indicating the difference in tumor growth increase magnitudes corresponding to the first and second subsequent tumor growth magnitudes.

Upon obtaining the subsequent tumor growth magnitude discrepancy, a lower discrepancy indicates that the difference in tumor growth increase magnitudes simulated by the similar first digital twin model and the target similar second digital twin model is smaller, implying a better simulation effect on the tumor growth process. Conversely, a higher discrepancy suggests a larger difference in tumor growth increase magnitudes simulated by these models, indicating a poorer simulation effect. Therefore, by recommending the recommended treatment decision directions in ascending order based on the subsequent tumor growth magnitude discrepancies of multiple predictive treatment decision model sets, the reliability of the recommended treatment decision directions is enhanced.

The beneficial effect of the aforementioned implementation method is that by recommending the recommended treatment decision directions in ascending order based on the subsequent tumor growth magnitude discrepancies of multiple predictive treatment decision model sets, the reliability of the recommended treatment decision directions is improved.

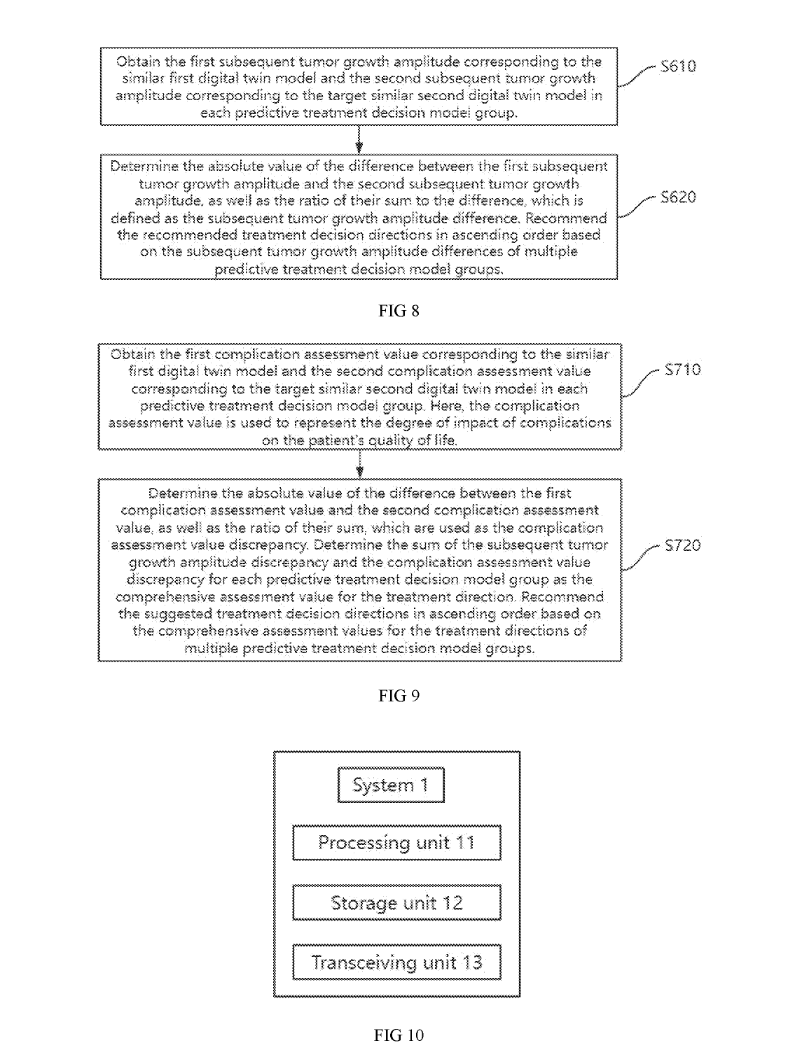

FIG. 9 is a flowchart of the seventh tumor growth prediction method based on digital twins provided in an embodiment of this application. As shown in FIG. 9, the aforementioned method further includes steps S710 to S720, which will be specifically described below.

S710: Obtain the first complication assessment value corresponding to the similar first digital twin model and the second complication assessment value corresponding to the target similar second digital twin model within each predictive treatment decision model set. Here, the complication assessment value is used to represent the degree of impact of complications on the patient's quality of life.

When evaluating tumor treatment directions, it is also possible to obtain the first complication assessment value corresponding to the similar first digital twin model and the second complication assessment value corresponding to the target similar second digital twin model within each predictive treatment decision model set. Subsequently, the first and second complication assessment values can be used to consider complications during tumor treatment for patients, thereby reducing the impact of complications on the patient's quality of life. Here, the complication assessment value is used to represent the degree of impact of complications on the patient's quality of life.

For example, the first complication assessment value corresponding to the similar first digital twin model and the second complication assessment value corresponding to the target similar second digital twin model within each predictive treatment decision model set can be determined based on empirical evidence related to the treatment direction.

For example, the complication assessment value can be represented by the sum of the impact scores of all complications on daily life.

S720: Determine the absolute value of the difference between the first and second complication assessment values, as well as the ratio of this difference to the sum of the first and second complication assessment values, and designate this ratio as the complication assessment value discrepancy. Calculate the sum of the subsequent tumor growth magnitude discrepancy and the complication assessment value discrepancy for each predictive treatment decision model set, and designate this sum as the comprehensive treatment direction assessment value. Recommend the recommended treatment decision directions in ascending order based on the comprehensive treatment direction assessment values of multiple predictive treatment decision model sets.

After obtaining the first and second complication assessment values, the absolute value of the difference between them, along with the ratio of this difference to their sum, can be calculated and designated as the complication assessment value discrepancy, which represents the difference between the first and second complication assessment values.

After obtaining the complication assessment value discrepancy, the sum of the subsequent tumor growth magnitude discrepancy and the complication assessment value discrepancy for each predictive treatment decision model set can be calculated and designated as the comprehensive treatment direction assessment value. This value represents a comprehensive assessment from the perspectives of tumor growth magnitude discrepancy and complication discrepancy. A higher comprehensive treatment direction assessment value indicates poorer simulation effects of the predictive treatment decision model set, while a lower value indicates better simulation effects. Subsequently, the recommended treatment decision directions can be recommended in ascending order based on the comprehensive treatment direction assessment values of multiple predictive treatment decision model sets, thereby recommending the treatment decision directions of predictive treatment decision model sets with better simulation effects to doctors and improving the reliability of the recommended treatment decision directions.

The beneficial effect of the aforementioned implementation method is that the complication assessment value discrepancy represents the difference between the first and second complication assessment values. By calculating the sum of the subsequent tumor growth magnitude discrepancy and the complication assessment value discrepancy for each predictive treatment decision model set as the comprehensive treatment direction assessment value, and recommending the recommended treatment decision directions in ascending order based on these values, the treatment decision directions of predictive treatment decision model sets with better simulation effects can be recommended to doctors, thereby improving the reliability of the recommended treatment decision directions.

This application embodiment also provides a tumor growth prediction system based on digital twins, which includes units for executing any of the aforementioned methods.

FIG. 10 is a schematic diagram of the logical structure of a tumor growth prediction system based on digital twins provided in an embodiment of this application. As shown in FIG. 10, the system 1 in this embodiment includes a processing unit 11, a storage unit 12, and a transceiver unit 13. The processing unit 11 is responsible for processing data, the storage unit 12 is used for storing data, and the transceiver unit 13 is used for transmitting and receiving data. The processing unit 11, storage unit 12, and transceiver unit 13 cooperate with each other to implement the aforementioned methods. The beneficial effects of this application embodiment have already been described in the aforementioned methods and will not be repeated here.

It should be noted that the information exchange and execution processes among the aforementioned devices/units are based on the same concept as the method embodiments of this application. Their specific functions and the technical effects they bring can be specifically referred to in the method embodiment section and will not be repeated here.

Those skilled in the field can clearly understand that, for the convenience and conciseness of description, the division of functional units and modules is merely illustrative. In practical applications, the aforementioned functions can be allocated to different functional units and modules as needed. That is, the internal structure of the device can be divided into different functional units or modules to accomplish all or part of the functions described above. The functional units and modules in the embodiments can be integrated into a single processing unit, or they can exist as separate physical entities, or two or more units can be integrated into one unit. These integrated units can be implemented in hardware form or as software functional units. Additionally, the specific names of the functional units and modules are merely for the purpose of mutual distinction and do not limit the scope of protection of this application. The specific working processes of the units and modules in the system can refer to the corresponding processes in the aforementioned method embodiments and will not be repeated here.

If the integrated unit is implemented in the form of a software functional unit and sold or used as an independent product, it can be stored in a computer-readable storage medium. Based on this understanding, all or part of the processes of the methods in the aforementioned embodiments of this application can be completed by instructing relevant hardware through a computer program. The computer program can be stored in a computer-readable storage medium. When executed by a processor, the computer program can implement the steps of each method embodiment described above. Here, the computer program includes computer program code, which can be in the form of source code, object code, executable files, or certain intermediate forms, etc. The computer-readable medium can include at least: any entity or device capable of carrying computer program code to a photographing device/terminal equipment, recording media, computer memory, read-only memory (ROM), random access memory (RAM), electrical carrier signals, telecommunication signals, and software distribution media, such as USB drives, mobile hard drives, magnetic disks, or optical disks, etc. In some jurisdictions, according to legislation and patent practices, computer-readable media may not include electrical carrier signals and telecommunication signals.

In the aforementioned embodiments, each embodiment has its own emphasis, and parts not detailed or described in one embodiment can be referred to in the relevant descriptions of other embodiments.

Those of ordinary skill in the field can realize that the units and algorithm steps described in the examples disclosed in this application can be implemented through electronic hardware, or a combination of computer software and electronic hardware. Whether these functions are executed in hardware or software form depends on the specific application and design constraints of the technical solution. Professionals can use different methods to implement the described functions for each specific application, but such implementations should not be considered beyond the scope of this application.

In the embodiments provided by this application, it should be understood that the disclosed devices and methods can be implemented in other ways. For example, the device embodiments described above are merely illustrative. The division of modules or units is only a logical function division, and there may be other division methods in actual implementation. For example, multiple units or components can be combined or integrated into another system, or some characteristics can be ignored or not executed. Additionally, the coupling or direct coupling or communication connection between the displayed or discussed components can be indirect coupling or communication connection through some interfaces, devices, or units, and can be in electrical, mechanical, or other forms.

The units described as separate components may or may not be physically separated, and the components displayed as units may or may not be physical units, that is, they can be located in the same place or distributed across multiple network units. Some or all of the units can be selected according to actual needs to achieve the objectives of this embodiment.

The aforementioned embodiments are only used to illustrate the technical solutions of this application and do not limit them. Although this application has been described in detail with reference to the aforementioned embodiments, those of ordinary skill in the field should understand that they can still make modifications to the technical solutions described in the aforementioned embodiments or replace some of the technical characteristics with equivalent ones. These modifications or replacements do not deviate from the spirit and scope of the technical solutions of the various embodiments of this application and should be included within the protection scope of this application.

1. A digital twin-based tumor growth prediction method, characterized by comprising:
constructing a digital twin database of the patient's multimodal pathological data, which includes brain imaging data, clinical data, physiological data, blood-brain barrier data, drug interaction data, and dynamic microenvironment data;
establishing a first digital twin model integrating brain imaging, clinical, and physiological data to reflect tumor growth patterns and evolution rates across different stages; and creating a second digital twin model incorporating blood-brain barrier data, drug interactions, and dynamic microenvironment data to characterize treatment microenvironments during various disease phases;
the first model generates initial treatment predictions based on the patient's treatment plan, while the second model produces secondary treatment predictions under the same protocol. Through treatment plan refinement modeling, the final optimized treatment strategy is determined by synthesizing the three-stage predictions and corresponding therapeutic adjustments.

2. The method according to claim 1, characterized in that the method further comprises:
determining a patient's blood-brain barrier characteristics through a blood-brain barrier detection model based on the patient's brain imaging data and blood-brain barrier data; and
determining a revised treatment plan for the patient through a treatment plan revision model based on the patient's treatment plan, first treatment prediction results, second treatment prediction results, and blood-brain barrier characteristics.

3. The method according to claim 2, characterized in that the method further comprises:
constructing drug concentration gradient brain imaging data corresponding to the patient's brain imaging data by integrating the patient's brain imaging data, blood-brain barrier data, and dynamic microenvironment data; and
establishing a first digital twin model comprising the patient's brain imaging data, clinical data, and physiological data, including: creating a first digital twin model integrating the patient's drug concentration gradient brain imaging data, clinical data, and physiological data.

4. The method according to claim 3, characterized in that the method further comprises:
determining normal tissue regions and tumor-infiltrated regions in the patient's brain imaging data, and acquiring corresponding normal tissue characteristics for the normal tissue regions and infiltration region characteristics for the tumor-infiltrated regions;
constructing a normal tissue drug concentration gradient brain imaging dataset based on the patient's normal tissue regions, normal tissue characteristics, blood-brain barrier data, and dynamic microenvironment data; constructing an infiltration region drug concentration gradient brain imaging dataset based on the patient's tumor-infiltrated regions, infiltration region characteristics, blood-brain barrier data, and dynamic microenvironment data;
establishing a first digital twin model integrating the patient's brain imaging data, clinical data, and physiological data, comprising: creating a first digital twin model incorporating the normal tissue drug concentration gradient brain imaging dataset, infiltration region drug concentration gradient brain imaging dataset, clinical data, and physiological data.

5. The method according to claim 4, characterized in that the method further comprises:
determining blood-brain barrier correction data through the first digital twin model using the patient's blood-brain barrier data, wherein the corrected data characterizes variations in the blood-brain barrier data;
constructing a second digital twin model integrating the patient's blood-brain barrier data, drug interaction data, and dynamic microenvironment data, comprising: creating a second digital twin model incorporating the blood-brain barrier correction data, drug interaction data, and dynamic microenvironment data.

6. The method according to claim 5, characterized in that the method further comprises:
obtaining normal blood-brain barrier characteristics corresponding to normal tissue regions and infiltrated blood-brain barrier characteristics corresponding to tumor-infiltrated regions; determining normal blood-brain barrier data for normal tissue regions based on the patient's blood-brain barrier data and brain imaging data of normal tissue regions with their corresponding blood-brain barrier characteristics; determining infiltrated blood-brain barrier data for tumor-infiltrated regions based on the patient's blood-brain barrier data and brain imaging data of tumor-infiltrated regions with their corresponding blood-brain barrier characteristics;
establishing first digital twin models to determine corrected blood-brain barrier data for normal tissue regions using the patient's normal blood-brain barrier data;
establishing second digital twin models to determine corrected blood-brain barrier data for infiltrated regions using the patient's infiltrated blood-brain barrier data; constructing a second digital twin model integrating the patient's blood-brain barrier data, drug interaction data, and dynamic microenvironment data, including: creating a second digital twin model encompassing corrected blood-brain barrier data for normal tissue regions, corrected blood-brain barrier data for infiltrated regions, drug interaction data, and dynamic microenvironment data.

7. The method according to claim 6, characterized in that the method further comprises:
obtaining multiple similar first digital twin models of the first digital twin model and multiple similar second digital twin models of the second digital twin model; acquiring treatment decision directions corresponding to the multiple similar first digital twin models and subsequent first digital twin models corresponding to the treatment decision directions; and obtaining treatment decision directions corresponding to the multiple similar second digital twin models and subsequent second digital twin models corresponding to the treatment decision directions, the treatment decision directions include aggressive treatment approaches and conservative treatment approaches;
among the multiple similar second digital twin models, determine target similar second digital twin models corresponding to the multiple similar first digital twin models based on parameter similarity of brain imaging data in the similar first digital twin models and blood-brain barrier data in the similar second digital twin models;
when the treatment decision direction of the similar first digital twin model matches that of the target similar second digital twin model, designate the similar first digital twin model and the target similar second digital twin model as a predicted treatment decision model group, and recommend the treatment decision direction corresponding to this group as the suggested treatment decision direction.

8. The method according to claim 7, characterized in that the method further comprises:
obtaining the first subsequent tumor growth amplitude corresponding to each similar first digital twin model in the group of predictive treatment decision models and the second subsequent tumor growth amplitude corresponding to the target similar second digital twin model;
determining the absolute value of the difference between the first subsequent tumor growth amplitude and the second subsequent tumor growth amplitude, as well as the ratio of their sum, as the subsequent tumor growth amplitude difference; recommending a suggested treatment decision direction based on the subsequent tumor growth amplitude differences of multiple groups of predictive treatment decision models ranked from low to high.

9. The method according to claim 8, characterized in that the method further comprises:
obtaining the first complication assessment value corresponding to each similar first digital twin model in the group of predictive treatment decision models and the second complication assessment value corresponding to the target similar second digital twin model; wherein the complication assessment value is used to characterize the impact of complications on the patient's quality of life;
determining the absolute value of the difference between the first complication assessment value and the second complication assessment value, as well as the ratio of their sum, as the complication assessment value difference; calculating the sum of the subsequent tumor growth amplitude difference and the complication assessment value difference for each group of predictive treatment decision models as the comprehensive treatment direction evaluation value; recommending a suggested treatment decision direction based on the comprehensive treatment direction evaluation values ranked from low to high across multiple groups of predictive treatment decision models.

10. A tumor growth prediction system based on digital twin, characterized in that the system comprises a unit for performing the method according to claim 9.
